(19) United States
(12) Patent Application Publication
(10) Pub. No.: US 2025/0046428 A1
(43) Pub. Date: Feb. 6, 2025

(54) BASELINE IMAGE GENERATION FOR DIAGNOSTIC APPLICATIONS

(71) Applicant: KONINKLIJKE PHILIPS N.V., EINDHOVEN (NL)

(72) Inventors: AXEL SAALBACH, HAMBURG (DE); TIM PHILIPP HARDER, AHRENSBURG (DE); THOMAS BUELOW, GROSSHANSDORF (DE); ANDRE GOOSSEN, ELDENA (DE); SVEN KROENKE-HILLE, HAMBURG (DE); JENS VON BERG, HAMBURG (DE); MICHAEL GRASS, BUCHHOLZ IN DER NORDHEIDE (DE)

(21) Appl. No.: 18/717,494

(22) PCT Filed: Dec. 6, 2022
(86) PCT No.: PCT/EP2022/084488

§ 371 Date: Jun. 7, 2024

Related U.S. Application Data
(60) Provisional application No. 63/288,030, filed on Dec. 10, 2021.

(51) Int. Cl. G16H 30/40; G06F 3/04842; G06T 5/77; G06V 10/82; G16H 30/20; G16H 50/20; G16H 50/70

(52) U.S. Cl. CPC G16H 30/40; G06F 3/04842; G06T 5/77; G06V 10/82; G16H 30/20; G16H 50/20; G16H 50/70

(57) ABSTRACT

Technology provides baseline images for diagnostic applications, including receiving a diagnostic image relating to a condition of a patient, the diagnostic image reflecting one of a normal state or an abnormal state of the condition, and generating a baseline image via a neural network using the diagnostic image, where the neural network is trained to generate a prediction of the diagnostic image reflecting a normal state of the condition. The neural network can include a generative adversarial network (GAN) trained only on image data with a normal state of the condition, where generating the baseline image includes an optimization process to maximize a similarity between the diagnostic image and a response of the GAN. Generating the baseline image can include selecting a portion of the diagnostic image, and adjusting a relevance weighting to be applied to the selected portion of the diagnostic image in the optimization process.

### FIELD OF THE INVENTION

Embodiments generally relate to computing technology. More particularly, embodiments relate to generating baseline images for diagnostic applications.

### BACKGROUND OF THE INVENTION

Medical professionals employ imaging of various modalities-such as, for example, X-ray, ultrasound, computed tomography (CT), and magnetic resonance imaging (MRI)—for medical diagnostic purposes. In order to assess the severity of a finding from a diagnostic image (i.e., a clinical image of a patient), a “normal” reference image for the same patient can be used for comparison with the diagnostic image. Without a reference image for the patient, severity assessments based on a diagnostic image are inherently difficult. Likewise, automated systems that monitor medical conditions need reference data in order to detect temporal changes. However, in many practical applications, such reference images are typically not available, resulting in a negative impact on the quality and accuracy of diagnostic assessments and change detection.

### SUMMARY OF THE INVENTION

There may be, therefore, a need to improve medical diagnostic imaging in terms of providing a way to generate reference or baseline images. An object of the disclosed technology is solved by the subject-matter of the appended independent claims, wherein further embodiments are incorporated in the dependent claims, in the accompanying drawings and the following description.

Disclosed herein are methods, systems, and computer readable media to generate baseline images, using a machine learning architecture, for diagnostic applications. The disclosed technology helps improve the overall performance of diagnostic systems by generating baseline images to be used in diagnostic applications, providing an improved ability to assess severity of findings from diagnostic imaging and/or to detect and track significant changes in condition. As a result, the quality and accuracy of diagnostic assessments and change tracking is enhanced.

According to aspects of the disclosed technology, a baseline image is generated via a neural network based on a diagnostic image. The diagnostic image reflects one of a normal state or an abnormal state of a condition of a patient, and can be an image generated by a diagnostic imaging system of a variety of types or modalities, such as, e.g., by X-ray, ultrasound, computed tomography (CT), magnetic resonance imaging (MRI), or other imaging techniques. The neural network can be a convolutional neural network (CNN), a recurrent neural network (RNN), a generative adversarial network (GAN), or another type of neural network, can have a plurality of layers, and is trained to generate a prediction of the diagnostic image reflecting a normal state of the condition of the patient.

In accordance with one or more embodiments, a computer-implemented method comprises receiving a diagnostic image relating to a condition of a patient, the diagnostic image reflecting one of a normal state or an abnormal state of the condition, and generating a baseline image via a neural network using the diagnostic image, wherein the neural network is trained to generate a prediction of the diagnostic image reflecting a normal state of the condition.

In accordance with one or more embodiments, a computer-implemented system comprises a processor, and a memory coupled to the processor, the memory comprising instructions which, when executed by the processor, cause the diagnostic system to perform operations comprising receiving a diagnostic image relating to a condition of a patient, the diagnostic image reflecting one of a normal state or an abnormal state of the condition, and generating a baseline image via a neural network using the diagnostic image, wherein the neural network is trained to generate a prediction of the diagnostic image reflecting a normal state of the condition.

In accordance with one or more embodiments, at least one non-transitory computer readable storage medium comprises instructions which, when executed by a computing system, cause the computing system to perform operations comprising receiving a diagnostic image relating to a condition of a patient, the diagnostic image reflecting one of a normal state or an abnormal state of the condition, and generating a baseline image via a neural network using the diagnostic image, wherein the neural network is trained to generate a prediction of the diagnostic image reflecting a normal state of the condition.

The features, functions, and advantages of the disclosed technology can be achieved independently in various examples or can be combined in yet other examples, further details of which can be seen with reference to the following description and drawings.

### BRIEF DESCRIPTION OF THE DRAWINGS

The various advantages of the embodiments of the present disclosure will become apparent to one skilled in the art by reading the following specification and appended claims, and by referencing the following drawings, in which:

FIG. 1 provides a block diagram illustrating an overview of an example of a baseline image generation system according to one or more embodiments;

FIGS. 2A-2E provide diagrams illustrating an example of a baseline image generation system according to one or more embodiments;

FIGS. 3A-3B provide diagrams illustrating an example of a baseline image generation system with constrained optimization according to one or more embodiments;

FIG. 4 provides a block diagram illustrating an example of a baseline image generation system according to one or more embodiments;

FIGS. 5A-5C provide diagrams illustrating examples of training a neural network according to one or more embodiments;

FIGS. 6A-6C provide flow diagrams illustrating a method of baseline image generation according to one or more embodiments; and

FIG. 7 is a diagram illustrating an example of a computing system for use in a baseline image generation system according to one or more embodiments.

### DETAILED DESCRIPTION OF EMBODIMENTS

Disclosed herein are improved computing systems, methods, and computer readable media to generate baseline images, using a machine learning architecture, for diagnostic applications. The disclosed technology helps improve the overall performance of diagnostic systems by generating baseline images to be used in diagnostic applications, providing an improved ability to assess severity of findings from diagnostic imaging and/or to detect and track significant changes in condition. As a result, the quality and accuracy of diagnostic assessments and change tracking is enhanced.

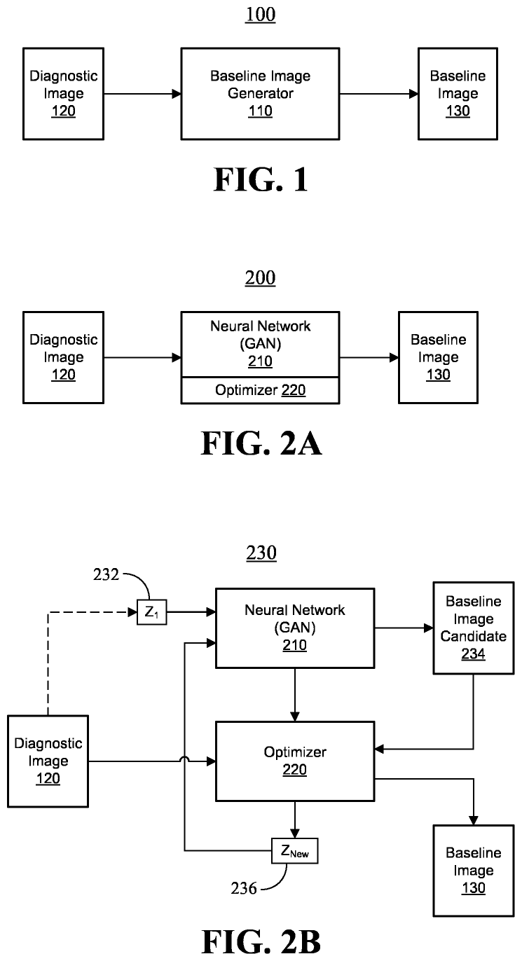

FIG. 1 provides a block diagram illustrating an overview of an example of a baseline image generation system 100 according to one or more embodiments, with reference to components and features described herein including but not limited to the figures and associated description. As shown in FIG. 1, the system 100 includes a baseline image generator 110 that is operable to generate a baseline image 130 based on a diagnostic image 120. The baseline image generator 110 can employ machine learning (ML) and/or deep learning or deep neural network (DNN) techniques.

The diagnostic image 120 can be an image generated by a diagnostic imaging system of a variety of types or modalities, such as, e.g., by X-ray, ultrasound, computed tomography (CT), magnetic resonance imaging (MRI), or other imaging techniques. The diagnostic image 120 typically will relate to a condition of a patient, and will reflect one of a normal state or an abnormal state of the condition. The baseline image 130 is a prediction, generated by the baseline image generator 110, of the diagnostic image reflecting a normal state of the condition. The baseline image 130 can then be used in comparing with the diagnostic image 120 to locate and identify areas in the diagnostic image 120 that can reflect an abnormal state of the condition. For example, the baseline image 130 can be displayed, along with a display of the diagnostic image 120, to a medical professional for evaluation, diagnosis, etc. The baseline image 130 can also be used in evaluating or determining the potential severity of an abnormal condition—e.g., by showing or highlighting the amount of difference between the baseline image 130 and the diagnostic image 120.

For example, the diagnostic image 120 can be a chest X-ray relating to a condition of a patient's lungs such as, e.g., with the presence or absence of indicia of pneumonia. The absence of indicia of pneumonia reflects a normal state of the condition, while the presence of indicia of pneumonia reflects an abnormal state of the condition. In this example, the system 100 can be used to process the diagnostic chest X-ray to generate a baseline image showing how the X-ray would appear if the patient did not exhibit indicia of pneumonia. The baseline image can then be used in comparing with the diagnostic image to locate and identify areas in the diagnostic image that can reflect an abnormal state of the condition (e.g., exhibiting indicia of pneumonia) and to show the severity of the indicia.

In another example, the diagnostic image 120 can be a chest X-ray relating to a patient's lungs which exhibits the presence or absence of indicia of COVID-19. In this example, the system 100 can be used to process the diagnostic chest X-ray to generate a baseline image showing how the X-ray would appear if the patient did not exhibit indicia of COVID-19. The baseline image can then be used in comparing with the diagnostic X-ray image to locate and identify areas in the diagnostic image that can reflect an abnormal state of the condition (e.g., exhibiting indicia of COVID-19), and the severity of the abnormal state of the condition.

In other examples, the diagnostic image can be a vascular image (e.g., ultrasound, CT scan, etc.) relating to a patient's arteries or veins. The system 100 can be used to process the diagnostic vascular image to generate a baseline image showing how the vascular image would appear if the patient had a normal state of the arteries/veins. The baseline image can be compared with the diagnostic vascular image and used to locate and identify areas in the diagnostic vascular image that can reflect an abnormal state of the condition of the arteries/veins—such as, e.g., stenosis or plaque in the arteries/veins, or an abdominal aortic aneurysm, or an infarct area of a stroke (e.g., showing collateralization in the infarct area)—and the severity of the abnormal state of the condition.

In one or more embodiments, the baseline image generator 110 can include components and features of neural network technology such as, e.g., a convolutional neural network (CNN), a recurrent neural network (RNN), a generative adversarial network (GAN), or another type of neural network, and can have a plurality of layers. A neural network employed in the baseline image generator 110 can be trained with training images reflecting a normal state of a condition (e.g., images which do not exhibit any abnormal or adverse findings). Once the neural network is trained using training images reflecting a normal state of a condition, the neural network can be used during the application phase (i.e., inference) to process a diagnostic image of a patient, where the diagnostic image can reflect one of a normal state or an abnormal state of the condition, and generate a baseline image that is similar to the diagnostic image but that reflects a normal state of the condition (or at least reflects an approximated appearance of a normal state of the condition) for the patient. For example, the neural network can be trained with chest X-ray images reflecting a normal state of a chest X-ray (e.g., absence of indicia of disease or other pathologies in the chest), and then used during the application phase (i.e., inference) to generate a baseline chest X-ray for the patient based on a diagnostic chest X-ray obtained for the patient.

In one or more embodiments, the neural network in the baseline image generator 110 can be trained to remove only a selected condition or conditions from a diagnostic image. For example, the neural network can be trained with image data reflecting a normal state for a particular condition but that otherwise reflect a variety of states for other conditions. When the baseline image generator 110 employs a neural network trained in such fashion and is then presented with a diagnostic image reflecting the particular condition, the baseline image generator 110 will process the diagnostic image and generate a baseline image reflecting a normal state as to the particular condition. Thus, for example, if the neural network is trained specifically with X-ray images reflecting a normal condition relating to COVID 19, the baseline image generator 110—when presented with a diagnostic X-ray image for a patient exhibiting both COVID-19 and chronic cardiomegaly—can generate a baseline image showing normal condition as to COVID-19 indicia without removing the indicia of cardiomegaly.

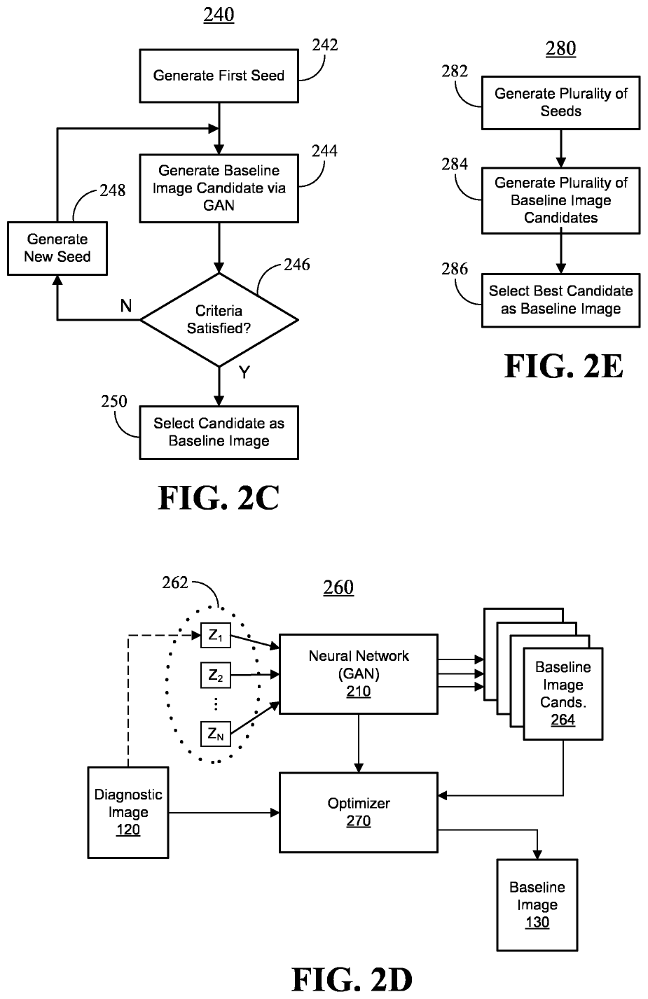

FIG. 2A provides a diagram illustrating an example of a baseline image generation system 200 according to one or more embodiments, with reference to components and features described herein including but not limited to the figures and associated description. The baseline image generation system 200 can correspond to the baseline image generation system 100 (FIG. 1, already discussed). As shown in FIG. 2A, the system 200 includes a neural network 210 that is a generative adversarial network (GAN). The GAN 210 includes or is accompanied by an optimizer 220. The system 200 is operable to generate a baseline image 130 based on the diagnostic image 120. The GAN 210/optimizer 220 can correspond to the baseline image generator 110 (FIG. 1, already discussed) and can be trained with images as discussed herein with reference to FIGS. 1 and 4A-4C.

When deployed as described herein, the GAN 210/optimizer 220 is operable to provide for the prediction of baseline (i.e., reference) image data using a large corpus of images such as, for example, images reflecting a normal state of a condition. The GAN 210 can include two main components, a generator and a discriminator. The generator network is trained in an iterative fashion using a large, representative set of images to generate new, “realistic” image data having similar statistics as the training images, while the discriminator network is trained at the same time to distinguish between generated and real images. During training, the discriminator causes the generator to produce better, more “realistic” images. As described above for the baseline image generator 110, the GAN 210 can be trained with images reflecting a normal state of a condition. When the GAN 210 is trained using images without pathologies, the GAN 210 is unable to reproduce a pathology contained in a diagnostic image, but instead provides a corresponding “normal” image (i.e., reflecting a normal state of the condition).

Once trained with images reflecting a normal state of a condition, the GAN 210/optimizer can be used to predict a diagnostic image of a patient (the diagnostic image reflecting a normal state or an abnormal state of the condition) and generate a baseline image 130 that is similar to the diagnostic image—but that reflects a normal state of the condition (or at least reflects an approximated appearance of a normal state of the condition) for the patient. Given a trained generator and an input diagnostic image, the system 200 causes a search for a baseline image 130 to be performed. During the application phase (i.e., inference), the GAN 210/optimizer 220 effectively performs an iterative search process to find a predicted image most similar to the current diagnostic image. Further details regarding embodiments of the baseline image generation system 200 are provided herein with reference to FIGS. 2B-2C. Further details regarding alternative embodiments of the baseline image generation system 200 are provided herein with reference to FIGS. 2D-2E.

Turning now to FIG. 2B, a diagram 230 illustrates operation of the baseline image generation system 200 according to one or more embodiments, with reference to components and features described herein including but not limited to the figures and associated description. A first seed Z₁ (label 232) is generated and input to the GAN 210. The seed Z₁ can be a scalar or a vector, and can be based on a random number or variable. In some embodiments, the seed Z₁ can be based on the diagnostic image 120 or on metadata associated with the diagnostic image 120 (or associated with the patient corresponding to the diagnostic image).

Based on the seed Z₁, the GAN 210 generates a baseline image candidate 234, which is input to the optimizer 220. The optimizer 220 evaluates the baseline image candidate 234, the based on the diagnostic image 120 and parameters and/or weights associated with the GAN 210, and determines whether criteria are met for selecting the baseline image. If the criteria are not met, a new seed Z_{New} (label 236) is generated and, as part of an iterative process, is provided as input to the GAN 210. The GAN 210 then generates a new baseline image candidate 234, which is input to the optimizer 220. The optimizer 220 then repeats the evaluation to determine whether criteria are met for selecting the baseline image. When the optimizer 220 determines that the criteria for selecting the baseline image are met, the most recent baseline image candidate 234 is selected as the generated baseline image 130.

In some embodiments, a backpropagation algorithm is employed by the optimizer 220 to determine the most similar image (e.g., match) that the generator can produce. To this end different objective functions can be employed, including the mean-squared-error (MSE) as a similarity measure to measure the intensity differences at a pixel level between the input diagnostic image 120 and each predicted image (i.e., each baseline image candidate 234) or a structural similarity index measure (SIIM), as well as a combination of different objective functions (incl. the discriminator response). In some embodiments, the optimizer 220 can employ an optimization technique such as, e.g., a gradient descent algorithm to iteratively adapt the seed Z to generate the new seed Z_{New} for the next iteration.

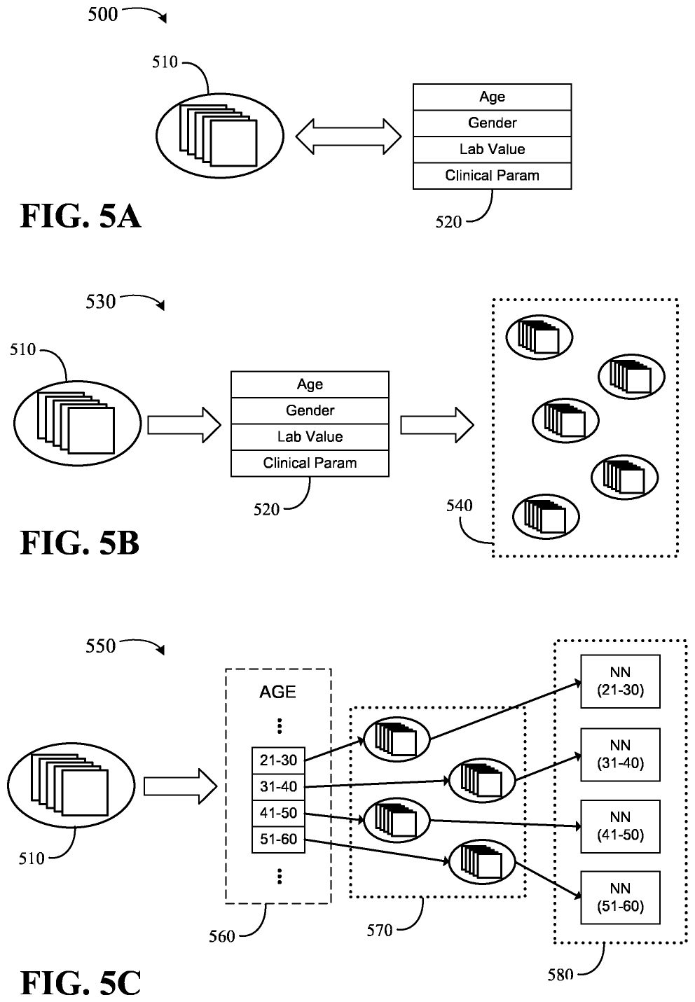

FIG. 2C provides a flow diagram of an example of a process 240 for operating the baseline image generation system 200 according to one or more embodiments, with reference to components and features described herein including but not limited to the figures and associated description. The process 240 can be an iterative process as discussed herein with reference to FIGS. 2A and 2B. Illustrated processing block 242 provides for generating a first seed (e.g., Z₁) for the GAN (e.g., GAN 210). The seed (e.g., first seed Z₁) can be a scalar or a vector, and can be based on a random number or variable. In some embodiments, the seed vector Z₁ can also be based on the input diagnostic image (e.g., the diagnostic image 120) or on metadata associated with the diagnostic image (or associated with the patient corresponding to the diagnostic image). For example, the seed vector Z₁ can consist of two components, a seed component and supplementary information. The supplementary information can include information about the diagnostic image (and its generation) such as, e.g., tube potential or intensity as well as exposure time and view position. Further (or alternatively), supplementary information can include context information relating to the diagnostic image such as, e.g., patient age, gender, lab values, and/or clinical parameters (as described herein with reference to FIGS. 5A-5C).

Illustrated processing block 244 provides for generating a baseline image candidate (e.g., the baseline image candidate 234) via the GAN, based on the seed as provided to the GAN. The baseline image candidate is generated by operating the GAN using the current seed. For example, in the first iteration through the process the current seed will be the first seed (e.g., Z₁) (block 242); in subsequent iterations the current seed will be the new seed (e.g., Z_{New}) (block 248).

Illustrated processing block 246 provides for determining whether criteria are met for selecting the baseline image. This determination can involve evaluation (e.g., by the optimizer 220) of the baseline image candidate, the diagnostic image (e.g., the diagnostic image 120), and parameters and/or weights associated with the GAN. Criteria for selecting the baseline image and terminating the iterative process can include one or more of the following:
(1) the baseline image candidate is sufficiently similar to the diagnostic image based on a similarity measure such as, e.g., a mean-squared-error (MSE) measure or a structural similarity index measure; for example, if the similarity measure exceeds a threshold similarity (or the difference is less than a threshold) the process can be terminated;
(2) a convergence of the solution is reached such that the next seed (Z_{New}) is sufficiently close to the most recent seed used to generate the most recent baseline image candidate (e.g., if the difference in seeds is less than a threshold, the process can be terminated); alternatively, a convergence can be reached if the most recent baseline image candidate is sufficiently close to the prior baseline image candidate (e.g., if the difference in baseline image candidates is less than a threshold, the process can be terminated); and/or
(3) a threshold number of iterations (e.g., loops through blocks 244-246 of the process 240) has been performed.

If the criteria are not met (No at block 246,) at processing block 248 a new seed (e.g., Z_{New}) is generated and the process returns to block 244.

When it is determined that the criteria for selecting the baseline image are met (Yes at block 246), the process continues to block 250. At processing block 250, the most recent baseline image candidate is selected as the generated baseline image 130.

Turning now to FIG. 2D, a diagram 260 illustrates operation of the baseline image generation system 200 according to one or more alternative embodiments, with reference to components and features described herein including but not limited to the figures and associated description. A plurality of seeds Z₁, Z₂, . . . , Z_{N} (label 262) is input to the GAN 210. Each of the seeds Z₁, Z₂, . . . , Z_{N} can be a scalar or a vector, and can be based on a random number or variable. In some embodiments, the seed Z₁ can be based on the diagnostic image 120 or on metadata associated with the diagnostic image 120 (or associated with the patient corresponding to the diagnostic image). In some embodiments, the other seeds Z₂, . . . , Z_{N} can be based on the seed Z₁. The seeds Z₁, Z₂, . . . , Z_{N} can be input sequentially to the GAN 210. For each of the seeds, the GAN 210 generates a baseline image candidate, which results in a plurality of baseline image candidates 264. Each of the plurality of baseline image candidates 264 is input to the optimizer 270, which measures the similarity between each of the baseline image candidates and the diagnostic image 120. Similarity measures as described with reference to FIGS. 2B-2C herein can be used by the optimizer 270. The candidate image of the plurality of baseline image candidates 264 having the greatest similarity to the diagnostic image 120 (e.g., having lowest MSE) is selected as the generated baseline image 130.

FIG. 2E provides a flow diagram of an example of a process 280 for operating the baseline image generation system 200 according to one or more alternative embodiments, with reference to components and features described herein including but not limited to the figures and associated description. Illustrated processing block 282 provides for generating a plurality of seeds (e.g., Z₁, Z₂, . . . , Z_{N}) for the GAN (e.g., GAN 210). Each of the seeds can be a scalar or a vector, and can be based on a random number or variable. In some embodiments, the first seed (e.g., Z₁) can be based on the input diagnostic image (e.g., the diagnostic image 120) or on metadata associated with the diagnostic image or the corresponding patient (for example, such as described herein with reference to FIG. 2C).

Illustrated processing block 284 provides for generating a plurality of baseline image candidates (e.g., baseline image candidates 264). Each baseline image candidate of the plurality of baseline image candidates is generated by operating the GAN using one of the seeds (e.g., Z₁, Z₂, . . . , Z_{N}). Illustrated processing block 286 provides for selecting the best baseline candidate from the plurality of baseline image candidates as the generated baseline image (e.g., baseline image 130). For example, the system 200 (e.g., via the optimizer 270) can measure the similarity between each of the baseline image candidates and the diagnostic image 120. Similarity measures as described with reference to FIGS. 2B-2C herein can be used. The candidate image of the plurality of baseline image candidates having the greatest similarity to the diagnostic image (e.g., having lowest MSE) is selected as the generated baseline image.

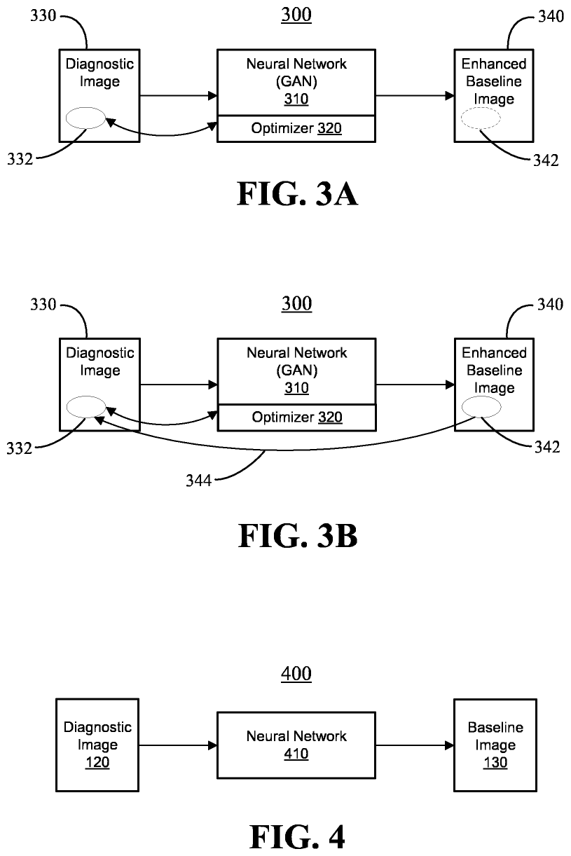

In some examples, a reference image obtained with the foregoing GAN approach might not always be optimal. A large pathology appearing in the diagnostic image could, in some circumstances, have a substantial impact on the match using a similarity measure. Additionally, the entire image might be subject to subtle differences compared to the diagnostic image. To address such concerns, a constrained optimization process is employed in some embodiments. FIGS. 3A-3B provide diagrams illustrating an example of a baseline image generation system 300 with constrained optimization according to one or more embodiments, with reference to components and features described herein including but not limited to the figures and associated description. The baseline image generation system 300 can correspond to the baseline image generation system 100 (FIG. 1, already discussed). As shown in FIG. 3A, the system 300 includes a neural network 310 that is a GAN. The GAN 310 includes or is accompanied by an optimizer 320. The GAN 310/optimizer 320 is operable to provide for the prediction of a baseline image 340, which can be an enhanced baseline image, based on a diagnostic image 330. The diagnostic image 330 can correspond (or be similar to) the diagnostic image (FIGS. 1 and 2A-2E, already discussed). The GAN 310 can correspond to the GAN 210 (FIGS. 2A-2E, already discussed). The optimizer 320 is a constrained optimizer and is similar to the optimizer 220 (FIGS. 2A-2C, already discussed) and/or to the optimizer 270 (FIGS. 2D-2E, already discussed). The system 300 operates similarly to the system 200 (FIGS. 2A-2E, already discussed), with differences as described herein.

As illustrated in FIG. 3A, the diagnostic image 330 includes a region 332 having a pathologic or suspicious area. The region 332 is selected or otherwise identified to the GAN 310/optimizer 320. The optimizer 320 is operable to de-emphasize or ignore the region 332 in performing the optimizing search for a baseline image (e.g., as a way to ignore a particular pathology when performing a search of baseline candidates). Stated another way, the optimizer 320 can be operable to restrict the optimization/similarity measure to the un-marked area(s) of the diagnostic image 330. For example, in performing an algorithm to locate the best match using a similarity measure (e.g., using a MSE function), the optimizer 320 can be configured to de-emphasize or exclude pixels in the region 332 in the input diagnostic image 330 when computing the MSE for each baseline image candidate. For example, the algorithm computing the similarity measure (e.g., MSE) can apply a relevance weighting to pixels in the region 332 such that the contribution of the region 332 to the similarity measure is reduced or eliminated. In some embodiments, alternatively the pixels in the region 332 can be set to a neutral or blank value (e.g., 0) or to a background value (e.g., an average value for all pixels in the image). Once the best match among baseline image candidates to the diagnostic image 330 is determined (where the region 332 has been ignored), such best match candidate selected as the generated baseline image 340. The baseline image 340 as generated by the system 300 can be considered enhanced to the extent it is generated with reduced or minimal impact caused by the pathology or suspicious area in the region 332. The baseline image 340 can be used in a manner as described with reference to the baseline image 130 (FIG. 1, already discussed).

In some embodiments, the baseline image 340 can have a region 342 corresponding to the same or similar location as the region 332 in the diagnostic image 330. For example, when displaying the baseline image 340, the region 342 can be de-emphasized or excluded from the display. In some embodiments, the region 342 can be de-emphasized or excluded from the display based on a selection (e.g., toggled on/off by an operator of the system 300)-effectively providing for an “eraser-like” function in the display process.

In some embodiments, the diagnostic image 330 can contain a relevant or highly relevant area, such that the region 332 should be emphasized or prioritized in the search of baseline candidate images. Accordingly, the algorithm computing the similarity measure (e.g., MSE) can apply a relevance weighting to pixels in the region 332 such that the contribution of the region 332 to the similarity measure is increased (e.g., prioritized). As one example, for an X-ray diagnostic image a region (or regions) corresponding to one or both lungs could be outlined and afforded greater weight for the GAN search process.

In some embodiments, the region 332 can be identified or selected, for example, by a computer-aided diagnosis application used to process the diagnostic image 330 (e.g., using segmentation). In some embodiments, the region 332 can be identified or selected through use of a selection tool provided via a graphical user interface (GUI). For example, a GUI can provide a selection functionality (such as, e.g., a box, lasso or a brush) for use by a medical professional to mark the region 332 as a pathologic or suspicious area in the diagnostic image 330.

Turning now to FIG. 3B, a further enhancement to the baseline image generation system 300 is illustrated. The baseline image 340 is shown with a region 342 corresponding to the same or similar location as the region 332 in the diagnostic image 330. The pixels of the baseline image 340 in the region 342 can be copied and substituted for the pixels of the diagnostic image 330 in the region 332, as shown at label 344. In effect, this provides the ability to “in-paint” a region (e.g., the region 332) in the diagnostic image 330 with a corresponding region of the baseline image 340 in order to provide a clearer basis for comparing the diagnostic image 330 and the baseline image 340 while ignoring or de-emphasizing a pathology that is not to be part of the diagnosis. For example, if a patient has a known preexisting condition that contributes a pathology to a region 332 of the diagnostic image 330 but the pathology is not to be part of a diagnosis of a new, unknown or suspect condition, the system 300 can not only de-emphasize or ignore the pathology in generating the baseline image 340 (as described with reference to FIG. 3A), but also in-paint the region 332 of the diagnostic image 330 with a corresponding region 342 (e.g., a “healthy” region without the known pathology) of the baseline image 340 to provide a stronger contrast between the diagnostic image 330 with the baseline image 340 as to effects other than the known pathology, thus enhancing the ability of a medical professional in using the diagnostic image 330 with the baseline image 340 for diagnosis of the new, unknown or suspect condition unknown or suspicious condition. Accordingly, by using this in-painting process, the impact of large or strongly localized pathologies on the predicted baseline image and diagnosis can be minimized, while at the same time most of the original diagnostic image data can be preserved.

In some examples, if the predicted baseline image is in-painted only in a user-selected region of the image (e.g., region 332), artifacts can occur at the boundary of this region. This can occur because the previously described constrained optimization does not involve a continuity prior on this boundary. Such a prior, however, can be implicitly realized as follows. During the iterative constrained optimization, the current predicted image is in-painted into the selected region of the diagnostic image. The resulting image is fed into the discriminator for predicting the probability of being a fake image, which is then added to the MSE loss as a prior term (with a corresponding weight factor). Since the discriminator has not “seen” any real images with the boundary artifacts during the GAN training, this term will prevent unrealistic boundary effects in the final in-painting.

FIG. 4 provides a block diagram illustrating an example of a baseline image generation system 400 according to one or more embodiments, with reference to components and features described herein including but not limited to the figures and associated description. The baseline image generation system 400 can correspond to the baseline image generation system 100 (FIG. 1, already discussed). As shown in FIG. 4, the system 400 includes a neural network 410 that is operable to generate a baseline image 130 (FIG. 1, already discussed) based on the diagnostic image 120 (FIG. 1, already discussed). The neural network 410 can include components and features of neural network technology such as, e.g., a convolutional neural network (CNN), a recurrent neural network (RNN), etc., and can have a plurality of layers. For example, the neural network 410 can include a fully convolutional network (such as, e.g., a U-Net type network). The neural network 410 can employ machine learning (ML) and/or deep learning or deep neural network (DNN) techniques.

The neural network 410 can be trained to translate images with an abnormal state to corresponding images with a normal state. Once trained, the neural network 410 can be used to translate a diagnostic image 120 (reflecting one of a normal state or an abnormal state of a condition of a patient) to a predicted baseline image 130 (representing, e.g., a prediction of the diagnostic image reflecting a normal state of the condition).

FIGS. 5A-5C provide diagrams illustrating examples of training a neural network according to one or more embodiments, with reference to components and features described herein including but not limited to the figures and associated description. Turning to FIG. 5A, a diagram 500 illustrates training data 510 to be used to train a neural network such as the GAN 210, the GAN 310, and/or the neural network 410. The training data 510 can include not only images but also data relating to the individuals who are the subjects of the training images. The data for the subject individuals can be captured as training metadata 520 (e.g., associated with each training image, or associated with the training set as a whole) including one or more characteristics such as, e.g., age of the individual who is the subject of the particular training image, gender of that individual, lab value(s) for that individual, and/or clinical parameter(s) relating to the image and/or that individual. Examples of lab values that can be captured and provided with the training images can include respiratory rate, blood pressure, pulse, pH levels, glucose levels, sodium levels, etc. Examples of clinical parameters that can be captured and provided with the training images can include patient history including, for example, history of liver disease, history of heart disease, history of chronic heart failure, etc. The training metadata 520 can be used during the training process, such that when the diagnostic image is presented along with data for the patient (e.g., for the patient age, patient gender, lab value(s), and/or clinical parameter(s) for the patient or test), the patient data will influence generation of the baseline image.

Turning now to FIG. 5B, a diagram 530 illustrates generating subsets of training image data. A full training image set 510 has associated training metadata 520 (such as described herein with reference to FIG. 5A). Based on the training metadata 520, the training data 510 can be divided, or allocated, into subsets of training data 540. For example, each subset of the subsets of training data 540 can correspond to images of a different population subset, where each population subset is associated with a particular range of one or more of the metadata characteristics of metadata 520 (e.g., one more of age, gender, lab value, or clinical parameter). In some embodiments, a separate neural network (e.g., the GAN 210, the GAN 310, and/or the neural network 410) can be trained using a respective subset of the training data subsets 540.

Turning now to FIG. 5C, a diagram 550 illustrates generating subsets of training image data (such as, e.g., as described herein with reference to FIG. 5B) for an example where the metadata 520 includes ranges of age data 560. In the example of FIG. 5C, age ranges can include a first range 21-30, a second range 31-40, a third range 41-50, a fourth range 51-60, and so forth. These age ranges can be used in dividing the training data 510 into subsets of training data 570, where each training data subset reflects (i.e., is associated with) one of the age ranges 560. Each of the raining data subsets 570 can be used to train a separate neural network to form a set of trained neural networks 580. In the example of FIG. 5C, the set of trained neural networks can include a first neural network corresponding to training data in the first range 21-30, a second neural network corresponding to training data in the second range 31-40, a third neural network corresponding to training data in the third range 41-50, and a fourth neural network corresponding to training data in the fourth range 51-60. Each trained neural network can be used to generate baseline images for diagnostic images for patients of the corresponding age range.

In some embodiments, a neural network (e.g., the GAN 210, the GAN 310, and/or the neural network 410) can be trained with selective removal-that is, trained with training data that includes images having conditions or pathologies (e.g., chronic conditions) other than a condition or pathology of interest. In some circumstances, using a neural network trained in this manner can result in generation of a baseline image representing a better match to a patient having a chronic condition but also an unknown severity of the condition or pathology of interest.

In some embodiments, a neural network (such as, e.g., the neural network 410 of FIG. 4, already discussed) can be trained with image pairs. The pairs of training images include image pairs from individuals in which, for a particular image pair, a first image of the pair reflects an individual with a particular condition in a normal state, and a second image of the pair reflects the same individual with a particular condition in an abnormal state. The neural network can then be trained to translate an image with an abnormal state to a corresponding image with a normal state by, e.g., minimizing the squared mean error of the neural network prediction to the target image of a normal state, based on the training image set. Training can employ, e.g., a stochastic gradient decent algorithm. Once trained, the neural network 410 can (as discussed herein with reference to FIG. 4) be used to translate a diagnostic image 120 (reflecting one of a normal state or an abnormal state of a condition of a patient) to a predicted baseline image 130 (representing, e.g., a prediction of the diagnostic image reflecting a normal state of the condition).

In some embodiments, a neural network (such as, e.g., the neural network 410 of FIG. 4, already discussed) can be trained in an unsupervised manner using training images obtained from image samples from healthy individuals. For example, the neural network 410 can be an encoder-decoder network trained in a manner to project the diagnostic image onto a subspace of images built from healthy sample images. Alternatively, in some embodiments the neural network (e.g., the neural network 410) can be an image translation model trained on an unpaired training data set consisting of a subset of images corresponding to normal state of condition and a second subset of images corresponding to an abnormal state of the condition. In this scenario, it is not required that for each image with a normal state there is an image with an abnormal state of the same patient. On such an unpaired training dataset, a cycle GAN model can be trained to translate images from of the first subset to a corresponding image of the second subset and vice versa in unsupervised manner by enforcing cyclic consistency of the image translations between the two subsets.

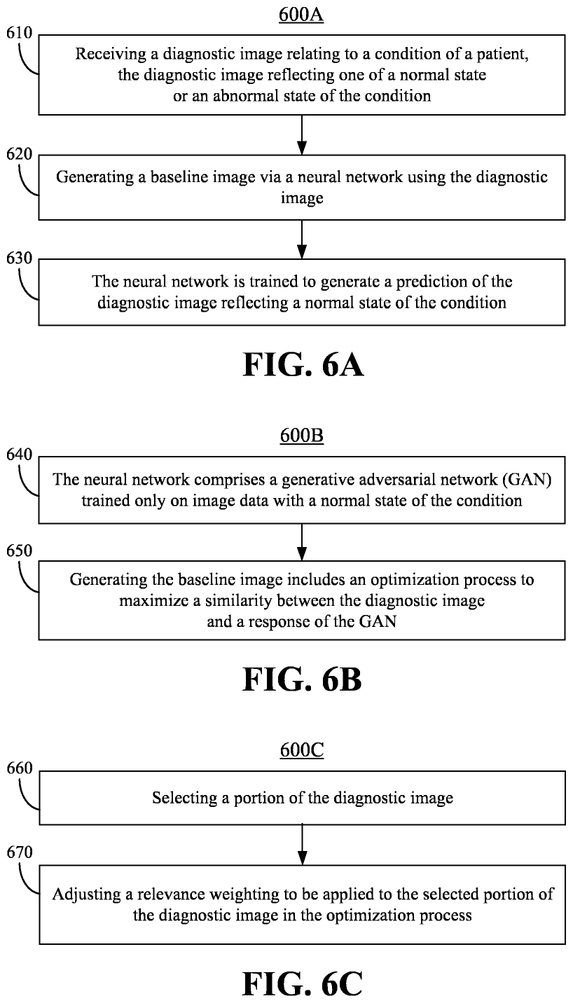

FIGS. 6A-6C provide flow diagrams illustrating a method 600 (components 600A, 600B and 600C) of baseline image generation according to one or more embodiments, with reference to components and features described herein including but not limited to the figures and associated description.

The method 600 and its components 600A, 600B and 600C can generally be implemented in the system 100 (FIG. 1, already discussed), in the system 200 (FIGS. 2A-2E, already discussed), in the system 300 (FIGS. 3A-3B, already discussed), and/or the system 400 (FIG. 4, already discussed). More particularly, the method 600 and its components 600A, 600B and 600C can be implemented as one or more modules in a set of program or logic instructions stored in a non-transitory machine- or computer-readable storage medium such as such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof.

For example, computer program code to carry out operations shown in the method 600 and its components 600A, 600B and 600C can be written in any combination of one or more programming languages, including an object oriented programming language such as JAVA, SMALLTALK, C++ or the like and conventional procedural programming languages, such as the “C” programming language or similar programming languages. Additionally, program or logic instructions might include assembler instructions, instruction set architecture (ISA) instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

Turning to FIG. 6A, the method 600A begins at illustrated processing block 610 by receiving a diagnostic image relating to a condition of a patient, the diagnostic image reflecting one of a normal state or an abnormal state of the condition. The diagnostic image can correspond to the diagnostic image 120 (FIGS. 1, 2A-2E and 4) and/or the diagnostic image 330 (FIGS. 3A-3B). Illustrated processing block 620 provides for generating a baseline image via a neural network using the diagnostic image. The neural network can correspond to the GAN 210 (FIGS. 2A-2E), the GAN 310 (FIGS. 3A-3B), and/or the neural network 410 (FIG. 4). The baseline image can correspond to the baseline image 130 (FIGS. 1, 2A-2E and 4), and/or the baseline image 340 (FIGS. 3A-3B). Illustrated processing block 630 provides that the neural network is trained to generate a prediction of the diagnostic image reflecting a normal state of the condition.

Turning now to FIG. 6B, illustrated processing block 640 of the method 600B provides that the neural network comprises a generative adversarial network (GAN) trained only on image data with a normal state of the condition. Illustrated processing block 650 provides that generating the baseline image includes an optimization process to maximize a similarity between the diagnostic image and a response of the GAN. The optimization process can correspond to some or all of the functions performed by the optimizer 220 (FIGS. 2A-2C, already discussed), the optimizer 270 (FIGS. 2D-2E, already discussed) and/or the optimizer 320 (FIGS. 3A-3B, already discussed). Illustrated processing block 650 can generally be substituted for at least a portion of illustrated processing block 620 (FIG. 6A, already discussed).

Turning now to FIG. 6C, illustrated processing block 660 of the method 600C provides for selecting a portion of the diagnostic image. Illustrated processing block 670 provides for adjusting a relevance weighting to be applied to the selected portion of the diagnostic image in the optimization process. The portion of the diagnostic image can correspond to region 332 (FIGS. 3A-3B, already discussed). In embodiments, illustrated processing blocks 660 and 670 can be included with at least part of illustrated processing block 650 (FIG. 6B, already discussed).

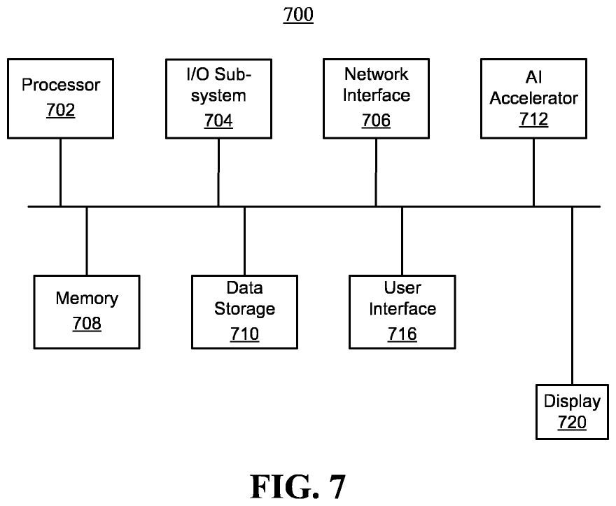

FIG. 7 is a diagram illustrating an example of a computing system 700 for use in a baseline image generation system (such as, e.g., the system 100 of FIG. 1, the system 200 of FIGS. 2A-2E, the system 300 of FIGS. 3A-3B, and/or the system 400 of FIG. 4) according to one or more embodiments, with reference to components and features described herein including but not limited to the figures and associated description. Although FIG. 7 illustrates certain components, the computing system 700 can include additional or multiple components connected in various ways. It is understood that not all examples will necessarily include every component shown in FIG. 7. As illustrated in FIG. 7, the computing system 700 includes one or more processors 702, an I/O subsystem 704, a network interface 706, a memory 708, a data storage 710, an artificial intelligence (AI) accelerator 712, a user interface 716, and/or a display 720. In some examples, the computing system 700 interfaces with a separate display. The computing system 700 can implement one or more components or features of the system 100, the system 200, the system 300, the system 400, the process 240, the process 280, the method 600 (including components 600A, 600B and/or 600C) and/or any of the components, features or methods described herein with reference to FIGS. 1, 2A-2E, 3A-3B, 4, 5A-5C, and/or 6A-6C.

The processor 702 can include one or more processing devices such as a microprocessor, a central processing unit (CPU), a fixed application-specific integrated circuit (ASIC) processor, a reduced instruction set computing (RISC) processor, a complex instruction set computing (CISC) processor, a field-programmable gate array (FPGA), etc., along with associated circuitry, logic, and/or interfaces. The processor 702 can include, or be connected to, a memory (such as, e.g., the memory 708) storing executable instructions and/or data, as necessary or appropriate. The processor 702 can execute such instructions to implement, control, operate or interface with any components or features of the system 100, the system 200, the system 300, the system 400, the process 240, the process 280, the method 600 (including components 600A, 600B and/or 600C) and/or any of the components, features or methods described herein with reference to FIGS. 1, 2A-2E, 3A-3B, 4, 5A-5C, and/or 6A-6C. The processor 702 can communicate, send, or receive messages, requests, notifications, data, etc. to/from other devices. The processor 702 can be embodied as any type of processor capable of performing the functions described herein. For example, the processor 702 can be embodied as a single or multi-core processor(s), a digital signal processor, a microcontroller, or other processor or processing/controlling circuit.

The I/O subsystem 704 includes circuitry and/or components suitable to facilitate input/output operations with the processor 702, the memory 708, and other components of the computing system 700.

The network interface 706 includes suitable logic, circuitry, and/or interfaces that transmits and receives data over one or more communication networks using one or more communication network protocols. The network interface 706 can operate under the control of the processor 702, and can transmit/receive various requests and messages to/from one or more other devices. The network interface 706 can include wired or wireless data communication capability; these capabilities support data communication with a wired or wireless communication network. The network interface 706 can support communication via a short-range wireless communication field, such as Bluetooth, NFC, or RFID. Examples of network interface 706 include, but are not limited to, one or more of an antenna, a radio frequency transceiver, a wireless transceiver, a Bluetooth transceiver, an ethernet port, a universal serial bus (USB) port, or any other device configured to transmit and receive data.

The memory 708 includes suitable logic, circuitry, and/or interfaces to store executable instructions and/or data, as necessary or appropriate, when executed, to implement, control, operate or interface with any components or features of the system 100, the system 200, the system 300, the system 400, the process 240, the process 280, the method 600 (including components 600A, 600B and/or 600C) and/or any of the components, features or methods described herein with reference to FIGS. 1, 2A-2E, 3A-3B, 4, 5A-5C, and/or 6A-6C. The memory 708 can be embodied as any type of volatile or non-volatile memory or data storage capable of performing the functions described herein, and can include a random-access memory (RAM), a read-only memory (ROM), write-once read-multiple memory (e.g., EEPROM), a removable storage drive, a hard disk drive (HDD), a flash memory, a solid-state memory, and the like, and including any combination thereof. In operation, the memory 708 can store various data and software used during operation of the computing system 700 such as operating systems, applications, programs, libraries, and drivers. Thus, the memory 708 can include at least one non-transitory computer readable medium comprising instructions which, when executed by the computing system 700, cause the computing system 700 to perform operations to carry out one or more functions or features of the system 100, the system 200, the system 300, the system 400, the process 240, the process 280, the method 600 (including components 600A, 600B and/or 600C) and/or any of the components, features or methods described herein with reference to FIGS. 1, 2A-2E, 3A-3B, 4, 5A-5C, and/or 6A-6C. The memory 708 can be communicatively coupled to the processor 702 directly or via the I/O subsystem 704.

The data storage 710 can include any type of device or devices configured for short-term or long-term storage of data such as, for example, memory devices and circuits, memory cards, hard disk drives, solid-state drives, non-volatile flash memory, or other data storage devices. The data storage 710 can include or be configured as a database, such as a relational or non-relational database, or a combination of more than one database. In some examples, a database or other data storage can be physically separate and/or remote from the computing system 700, and/or can be located in another computing device, a database server, on a cloud-based platform, or in any storage device that is in data communication with the computing system 700.

The artificial intelligence (AI) accelerator 712 includes suitable logic, circuitry, and/or interfaces to accelerate artificial intelligence applications, such as, e.g., artificial neural networks, machine vision and machine learning applications, including through parallel processing techniques. In one or more examples, the AI accelerator 712 can include a graphics processing unit (GPU). The AI accelerator 712 can implement one or more any components or features of the system 100, the system 200, the system 300, the system 400, the process 240, the process 280, the method 600 (including components 600A, 600B and/or 600C) and/or any of the components, features or methods described herein with reference to FIGS. 1, 2A-2E, 3A-3B, 4, 5A-5C, and/or 6A-6C, including one or more of the neural network 210 (FIGS. 2A-2B), the neural network 310 (FIGS. 3A-3B), and/or the neural network 410 (FIG. 4). In some examples the computing system 700 includes a second AI accelerator (not shown).

The user interface 716 includes code to present, on a display, information or screens for a user and to receive input (including commands) from a user via an input device. For example, the user interface 716 can provide a selection tool via a GUI for use in selecting the region 332 as described herein with reference to FIGS. 3A-3B.

The display 720 can be any type of device for presenting visual information, such as a computer monitor, a flat panel display, or a mobile device screen, and can include a liquid crystal display (LCD), a light-emitting diode (LED) display, a plasma panel, or a cathode ray tube display, etc. The display 720 can include a display interface for communicating with the display. In some examples, the display 720 can incorporate two or more physical displays. In some examples, the display 720 can include a display interface for communicating with a display external to the computing system 700. For example, the display 720 can display one or more of the diagnostic image 120, the diagnostic image 330, the baseline image 130, and/or the baseline image 340.

In embodiments, one or more of the illustrative components of the computing system 700 can be incorporated (in whole or in part) within, or otherwise form a portion of, another component. For example, the memory 708, or portions thereof, can be incorporated within the processor 702. As another example, the user interface 716 can be incorporated within the processor 702 and/or code in the memory 708. In some examples, the computing system 700 can be embodied as, without limitation, a mobile computing device, a smartphone, a wearable computing device, an Internet-of-Things device, a laptop computer, a tablet computer, a notebook computer, a computer, a workstation, a server, a multiprocessor system, and/or a consumer electronic device. In some examples, the computing system 700, or portion thereof, is implemented in one or more modules as a set of logic instructions stored in at least one non-transitory machine- or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality logic hardware using circuit technology such as, for example, application specific integrated circuit (ASIC), complementary metal oxide semiconductor (CMOS) or transistor-transistor logic (TTL) technology, or any combination thereof.

For example, computer program code to carry out operations by the system 700 can be written in any combination of one or more programming languages, including an object oriented programming language such as JAVA, SMALLTALK, C++ or the like and conventional procedural programming languages, such as the “C” programming language or similar programming languages. Additionally, program or logic instructions might include assembler instructions, instruction set architecture (ISA) instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

In embodiments, all or portions of the system 100, the system 200, the system 300, the system 400, and/or the system 700 can be implemented in, or integrated with, or otherwise combined with a diagnostic imaging system (such as, e.g., an X-ray imaging system, a PACS viewer or a diagnostic workstation). Additionally, all or portions of the system 100, the system 200, the system 300, the system 400, and/or the system 700 can be implemented in, or integrated with, or otherwise combined with a computer-aided diagnostic (CAD) system, including for temporal change monitoring.

Embodiments of each of the above systems, devices, components and/or methods, including the system 100, the system 200, the system 300, the system 400, the system 700, the process 240, the process 280, the method 600 (including components 600A, 600B and/or 600C) and/or any of the components, features or methods described herein with reference to FIGS. 1, 2A-2E, 3A-3B, 4, 5A-5C, 6A-6C and/or 7, and/or any other system components, can be implemented in hardware, software, or any suitable combination thereof. For example, hardware implementations can include configurable logic such as, for example, programmable logic arrays (PLAs), field programmable gate arrays (FPGAs), complex programmable logic devices (CPLDs), or fixed-functionality logic hardware using circuit technology such as, for example, application specific integrated circuit (ASIC), complementary metal oxide semiconductor (CMOS) or transistor-transistor logic (TTL) technology, or any combination thereof.

Alternatively, or additionally, all or portions of the foregoing systems and/or components and/or methods can be implemented in one or more modules as a set of program or logic instructions stored in a machine- or computer-readable storage medium such as RAM, ROM, PROM, firmware, flash memory, etc., to be executed by a processor or computing device. For example, computer program code to carry out the operations of the foregoing systems and/or components and/or methods can be written in any combination of one or more operating system (OS) applicable/appropriate programming languages, including an object-oriented programming language such as PYTHON, PERL, JAVA, SMALLTALK, C++, C# or the like and conventional procedural programming languages, such as the “C” programming language or similar programming languages.

### Additional Notes and Examples

Example 1 includes a computer-implemented method, comprising receiving a diagnostic image relating to a condition of a patient, the diagnostic image reflecting one of a normal state or an abnormal state of the condition, and generating a baseline image via a neural network using the diagnostic image, wherein the neural network is trained to generate a prediction of the diagnostic image reflecting a normal state of the condition.
Example 2 includes the method of Example 1, wherein the neural network comprises a generative adversarial network (GAN) trained only on image data with a normal state of the condition, and wherein generating the baseline image includes an optimization process to maximize a similarity between the diagnostic image and a response of the GAN.
Example 3 includes the method of Example 1 or 2, wherein generating the baseline image includes selecting a portion of the diagnostic image, and adjusting a relevance weighting to be applied to the selected portion of the diagnostic image in the optimization process.
Example 4 includes the method of Example 1, 2, or 3, wherein selecting a portion of the diagnostic image is performed via one or more of a computer-aided diagnosis application or a selection tool provided by a graphical user interface.
Example 5 includes the method of any of Examples 1-4, wherein a portion of the baseline image corresponding to the selected portion of the diagnostic image is used to in-paint the selected portion of the diagnostic image.
Example 6 includes the method of any of Examples 1-5, wherein the neural network is trained on one or more subsets of training data, wherein each subset of training data corresponds to images of a different population subset, each population subset associated with a particular range of one or more characteristics, wherein the one or more characteristics includes one or more of age, gender, lab value, or clinical parameter.
Example 7 includes the method of any of Examples 1-6, wherein the neural network is trained to remove a selected condition from training image data.
Example 8 includes the method of any of Examples 1-7, wherein the neural network is an image translation model trained on an unpaired training data set.
Example 9 includes a computing system, comprising a processor, and a memory coupled to the processor, the memory comprising instructions which, when executed by the processor, cause the computing system to perform operations comprising receiving a diagnostic image relating to a condition of a patient, the diagnostic image reflecting one of a normal state or an abnormal state of the condition, and generating a baseline image via a neural network using the diagnostic image, wherein the neural network is trained to generate a prediction of the diagnostic image reflecting a normal state of the condition.
Example 10 includes the computing system of Example 9, wherein the neural network comprises a generative adversarial network (GAN) trained only on image data with a normal state of the condition, and wherein generating the baseline image includes an optimization process to maximize a similarity between the diagnostic image and a response of the GAN.
Example 11 includes the computing system of Example 9 or 10, wherein generating the baseline image includes selecting a portion of the diagnostic image, and adjusting a relevance weighting to be applied to the selected portion of the diagnostic image in the optimization process, wherein selecting a portion of the diagnostic image is performed via one or more of a computer-aided diagnosis application or a selection tool provided by a graphical user interface, and wherein a portion of the baseline image corresponding to the selected portion of the diagnostic image is used to in-paint the selected portion of the diagnostic image.
Example 12 includes the computing system of Example 9, 10, or 11, wherein the neural network is trained on one or more subsets of training data, wherein each subset of training data corresponds to images of a different population subset, each population subset associated with a particular range of one or more characteristics, wherein the one or more characteristics includes one or more of age, gender, lab value, or clinical parameter.
Example 13 includes the computing system of any of Examples 9-12, wherein the neural network is trained to remove a selected condition from training image data.
Example 14 includes the computing system of any of Examples 9-13, wherein the neural network is an image translation model trained on an unpaired training data set.
Example 15 includes at least one non-transitory computer readable storage medium comprising instructions which, when executed by a computing system, cause the computing system to perform operations comprising receiving a diagnostic image relating to a condition of a patient, the diagnostic image reflecting one of a normal state or an abnormal state of the condition, and generating a baseline image via a neural network using the diagnostic image, wherein the neural network is trained to generate a prediction of the diagnostic image reflecting a normal state of the condition.
Example 16 includes the at least one non-transitory computer readable storage medium of Example 15, wherein the neural network comprises a generative adversarial network (GAN) trained only on image data with a normal state of the condition, and wherein generating the baseline image includes an optimization process to maximize a similarity between the diagnostic image and a response of the GAN.
Example 17 includes the at least one non-transitory computer readable storage medium of Example 15 or 16, wherein generating the baseline image includes selecting a portion of the diagnostic image, and adjusting a relevance weighting to be applied to the selected portion of the diagnostic image in the optimization process, wherein selecting a portion of the diagnostic image is performed via one or more of a computer-aided diagnosis application or a selection tool provided by a graphical user interface, and wherein a portion of the baseline image corresponding to the selected portion of the diagnostic image is used to in-paint the selected portion of the diagnostic image.
Example 18 includes the at least one non-transitory computer readable storage medium of Example 15, 16, or 17, wherein the neural network is trained on one or more subsets of training data, wherein each subset of training data corresponds to images of a different population subset, each population subset associated with a particular range of one or more characteristics, wherein the one or more characteristics includes one or more of age, gender, lab value, or clinical parameter.
Example 19 includes the at least one non-transitory computer readable storage medium of any of Examples 15-18, wherein the neural network is trained to remove a selected condition from training image data.
Example 20 includes the at least one non-transitory computer readable storage medium of any of Examples 15-19, wherein the neural network is an image translation model trained on an unpaired training data set.
Example 21 includes an apparatus comprising means for performing the method of any one of Examples 1-8.

Embodiments are applicable for use with all types of semiconductor integrated circuit (“IC”) chips. Examples of these IC chips include but are not limited to processors, controllers, chipset components, programmable logic arrays (PLAs), memory chips, network chips, systems on chip (SoCs), SSD/NAND controller ASICs, and the like. In addition, in some of the drawings, signal conductor lines are represented with lines. Some may be different, to indicate more constituent signal paths, have a number label, to indicate a number of constituent signal paths, and/or have arrows at one or more ends, to indicate primary information flow direction. This, however, should not be construed in a limiting manner. Rather, such added detail may be used in connection with one or more exemplary embodiments to facilitate easier understanding of a circuit. Any represented signal lines, whether or not having additional information, may actually comprise one or more signals that may travel in multiple directions and may be implemented with any suitable type of signal scheme, e.g., digital or analog lines implemented with differential pairs, optical fiber lines, and/or single-ended lines.

Example sizes/models/values/ranges may have been given, although embodiments are not limited to the same. As manufacturing techniques (e.g., photolithography) mature over time, it is expected that devices of smaller size could be manufactured. In addition, well known power/ground connections to IC chips and other components may or may not be shown within the figures, for simplicity of illustration and discussion, and so as not to obscure certain aspects of the embodiments. Further, arrangements may be shown in block diagram form in order to avoid obscuring embodiments, and also in view of the fact that specifics with respect to implementation of such block diagram arrangements are highly dependent upon the platform within which the embodiment is to be implemented, i.e., such specifics should be well within purview of one skilled in the art. Where specific details (e.g., circuits) are set forth in order to describe example embodiments, it should be apparent to one skilled in the art that embodiments can be practiced without, or with variation of, these specific details. Unless otherwise explicitly stated herein, the order of operations or steps described with reference to any of the processes or methods herein is not critical to the disclosed technology. The description is thus to be regarded as illustrative instead of limiting.

The term “coupled” may be used herein to refer to any type of relationship, direct or indirect, between the components in question, and may apply to electrical, mechanical, fluid, optical, electromagnetic, electromechanical or other connections, including logical connections via intermediate components (e.g., device A may be coupled to device C via device B). In addition, the terms “first”, “second”, etc. may be used herein only to facilitate discussion, and carry no particular temporal or chronological significance unless otherwise indicated.

As used in this application and in the claims, a list of items joined by the term “one or more of” may mean any combination of the listed terms. For example, the phrases “one or more of A, B or C” may mean A, B, C; A and B; A and C; B and C; or A, B and C. Furthermore, in the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the drawings, specification, and following claims.

1. A computer-implemented method, comprising:
receiving a diagnostic image relating to a condition of a patient, the diagnostic image reflecting one of a normal state or an abnormal state of the condition; and
generating a baseline image via a neural network using the diagnostic image;
wherein the neural network is trained to generate a prediction of the diagnostic image reflecting a normal state of the condition.

2. The method of claim 1, wherein the neural network comprises a generative adversarial network (GAN) trained only on image data with a normal state of the condition, and
wherein generating the baseline image includes an optimization process to maximize a similarity between the diagnostic image and a response of the GAN.

3. The method of claim 2, wherein generating the baseline image includes:
selecting a portion of the diagnostic image; and
adjusting a relevance weighting to be applied to the selected portion of the diagnostic image in the optimization process.

4. The method of claim 3, wherein selecting a portion of the diagnostic image is performed via one or more of a computer-aided diagnosis application or a selection tool provided by a graphical user interface.

5. The method of claim 3, wherein a portion of the baseline image corresponding to the selected portion of the diagnostic image is used to in-paint the selected portion of the diagnostic image.

6. The method of claim 1, wherein the neural network is trained on one or more subsets of training data, wherein each subset of training data corresponds to images of a different population subset, each population subset associated with a particular range of one or more characteristics, wherein the one or more characteristics includes one or more of age, gender, lab value, or clinical parameter.

7. The method of claim 1, wherein the neural network is trained to remove a selected condition from training image data.

8. The method of claim 1, wherein the neural network is an image translation model trained on an unpaired training data set.

9. A computing system comprising:
a processor; and
a memory coupled to the processor, the memory comprising instructions which, when executed by the processor, cause the computing system to perform operations comprising:
receiving a diagnostic image relating to a condition of a patient, the diagnostic image reflecting one of a normal state or an abnormal state of the condition; and
generating a baseline image via a neural network using the diagnostic image;
wherein the neural network is trained to generate a prediction of the diagnostic image reflecting a normal state of the condition.

10. The computing system of claim 9, wherein the neural network comprises a generative adversarial network (GAN) trained only on image data with a normal state of the condition, and
wherein generating the baseline image includes an optimization process to maximize a similarity between the diagnostic image and a response of the GAN.

11. The computing system of claim 10, wherein generating the baseline image includes:
selecting a portion of the diagnostic image; and
adjusting a relevance weighting to be applied to the selected portion of the diagnostic image in the optimization process,
wherein selecting a portion of the diagnostic image is performed via one or more of a computer-aided diagnosis application or a selection tool provided by a graphical user interface, and
wherein a portion of the baseline image corresponding to the selected portion of the diagnostic image is used to in-paint the selected portion of the diagnostic image.

12. The computing system of claim 9, wherein the neural network is trained on one or more subsets of training data, wherein each subset of training data corresponds to images of a different population subset, each population subset associated with a particular range of one or more characteristics, wherein the one or more characteristics includes one or more of age, gender, lab value, or clinical parameter.

13. The computing system of claim 9, wherein the neural network is trained to remove a selected condition from training image data.

14. The computing system of claim 9, wherein the neural network is an image translation model trained on an unpaired training data set.

15. At least one non-transitory computer readable storage medium comprising instructions which, when executed by a computing system, cause the computing system to perform operations comprising:
receiving a diagnostic image relating to a condition of a patient, the diagnostic image reflecting one of a normal state or an abnormal state of the condition; and
generating a baseline image via a neural network using the diagnostic image;
wherein the neural network is trained to generate a prediction of the diagnostic image reflecting a normal state of the condition.

16. The at least one non-transitory computer readable storage medium of claim 15, wherein the neural network comprises a generative adversarial network (GAN) trained only on image data with a normal state of the condition, and
wherein generating the baseline image includes an optimization process to maximize a similarity between the diagnostic image and a response of the GAN.

17. The at least one non-transitory computer readable storage medium of claim 16, wherein generating the baseline image includes:
selecting a portion of the diagnostic image; and
adjusting a relevance weighting to be applied to the selected portion of the diagnostic image in the optimization process,
wherein selecting a portion of the diagnostic image is performed via one or more of a computer-aided diagnosis application or a selection tool provided by a graphical user interface, and
wherein a portion of the baseline image corresponding to the selected portion of the diagnostic image is used to in-paint the selected portion of the diagnostic image.

18. The at least one non-transitory computer readable storage medium of claim 15, wherein the neural network is trained on one or more subsets of training data, wherein each subset of training data corresponds to images of a different population subset, each population subset associated with a particular range of one or more characteristics, wherein the one or more characteristics includes one or more of age, gender, lab value, or clinical parameter.

19. The at least one non-transitory computer readable storage medium of claim 15, wherein the neural network is trained to remove a selected condition from training image data.

20. The at least one non-transitory computer readable storage medium of claim 15, wherein the neural network is an image translation model trained on an unpaired training data set.
